(19) United States
(12) Patent Application Publication
(10) Pub. No.: US 2006/0051865 A1
(43) Pub. Date: Mar. 9, 2006

(54) Systems and methods for isolating stromal cells from adipose tissue and uses thereof

(76) Inventors: Joel C. Higgins, Claypool, IN (US); Michael Dean Leach, Warsaw, IN (US); Felipe Palacios, Pearland, TX (US); Nicolaas Vermeulen, North Manchester, IN (US)

Correspondence Address: HARNESS, DICKEY & PIERCE, P.L.C., P.O. BOX 828, BLOOMFIELD HILLS, MI, 48303, US

(21) Appl. No.: 11/217,087

(22) Filed: Aug. 31, 2005

Related U.S. Application Data
(60) Provisional application No. 60/606,090, filed on Aug. 31, 2004.

(51) Int. Cl. C12N 5/08

(52) U.S. Cl. 435/366

(57) ABSTRACT

Methods of isolating cells from adipose tissue that have potential to differentiate into cells of mesenchymal origin, including cells of chondrogenic, osteogenic, adipogenic, and/or myogenic origin, comprising: (a) subjecting adipose tissue to an electromagnetic, sonic, or other wave energy source; and (b) centrifuging the tissue to form a pellet comprising stem cells. In various embodiments, the method is carried out without any enzymatic digestion of the adipose tissue. In other embodiments, the method additionally comprises enzymatically digesting the tissue. In various embodiments, methods comprise subjecting the tissue to ultrasonic energy. In some embodiments, the method does not comprise enzymatic digestion of the adipose tissue. In other embodiments, the method additionally comprises enzymatically digesting the tissue. Methods are also provided for intraoperative harvest and delivery of autologous stem cells to the site of acute or chronic wounds. In one embodiment, an autologous supply of stem cells is provided for operative use.

### CROSS-REFERENCE TO RELATED APPLICATIONS

This application claims the benefit of U.S. Provisional Application No. 60/606,090, filed on Aug. 31, 2004, which is herein incorporated by reference in its entirety.

### BACKGROUND

The present disclosure relates to methods for deriving stem cells from adipose tissue.

Recent studies suggest that human adipose tissue contains pluripotent or multipotent stem cells similar to bone marrow derived stem cells. These cells have been termed adipose derived adult stem (ADAS) cells, as they are self-renewing and can be induced to various mesenchymal lineages, including chondrocytes, adipocytes, osteoplasts, myocytes, and cardiomyocytes. It has also been reported that ADAS cells can be induced to undergo morphologic and phenotypic changes consistent with neuronal differentiation.

Because adipose tissue is plentiful and easily harvested in large quantity under local anesthesia with little patient discomfort, it has potential to provide an alternative source of stem cells for tissue regeneration and engineering.

Known methods of isolating stem cells from adipose tissue include a step of enzymatic digestion such as with collagenase. However, the enzymatic digestion and other steps are time-consuming and sensitive to various conditions such as temperature pH, and purity of reagents.

### SUMMARY

The present disclosure provides methods of isolating cells from adipose tissue that have potential to differentiate into cells of mesenchymal origin, including cells of chondrogenic, osteogenic, adipogenic, and/or myogenic origin. Methods include:
(a) subjecting adipose tissue to an electromagnetic, sonic, or other wave energy source; and
(b) centrifuging the tissue to form a pellet comprising stem cells.
   In various embodiments, the methods comprise subjecting the tissue to sonic energy, preferably ultrasonic energy. In some embodiments, the method does not comprise enzymatic digestion of the adipose tissue.

In various embodiments, the adipose tissue is harvested such as by liposuction, and the lipoaspirate is exposed to ultrasonic energy to break up the connective matrix. Following exposure to ultrasound, the sonicated tissue is centrifuged and adult stem cells are recovered from the pellet. In various embodiments, methods are provided for intraoperative harvest and delivery of autologous stem cells to the site of acute or chronic wounds such as surgical incisions, diabetic ulcers, bed sores, and the like. In one embodiment, an autologous supply of stem cells is provided for operative use.

Further areas of applicability of the present invention will become apparent from the detailed description provided hereinafter. It should be understood that the detailed description and specific examples are intended for purposes of illustration only and are not intended to limit the scope of the invention.

### BRIEF DESCRIPTION OF THE DRAWINGS

FIG. 1 is an illustration of a device adapted for use in isolating ADAS cells from adipose tissue.

It should be noted that this figure is intended to show the general characteristics of devices among those useful in this invention, for the purpose of the description of such embodiments herein. This figure may not precisely reflect the characteristics of any given embodiment, and is not necessarily intended to define or limit specific embodiments within the scope of this invention.

### DESCRIPTION

The headings (such as “Introduction” and “Summary,”) used herein are intended only for general organization of topics within the disclosure of the invention, and are not intended to limit the disclosure of the invention or any aspect thereof. In particular, subject matter disclosed in the “Introduction” may include aspects of technology within the scope of the invention, and may not constitute a recitation of prior art. Subject matter disclosed in the “Summary” is not an exhaustive or complete disclosure of the entire scope of the invention or any embodiments thereof.

The description and specific examples, while indicating embodiments of the invention, are intended for purposes of illustration only and are not intended to limit the scope of the invention. Moreover, recitation of multiple embodiments having stated features is not intended to exclude other embodiments having additional features, or other embodiments incorporating different combinations of the stated features. Specific examples are provided for illustrative purposes of how to make, use and practice the compositions and methods of this invention and, unless explicitly stated otherwise, are not intended to be a representation that given embodiments of this invention have, or have not, been made or tested.

As used herein, the words “preferred” and “preferably” refer to embodiments of the invention that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, the word “include,” and its variants, is intended to be non-limiting, such that recitation of items in a list is not to the exclusion of other like items that may also be useful in the materials, compositions, devices, and methods of this invention.

A method for isolating or recovering adult stem cells from adipose tissue includes the steps of subjecting the adipose tissue to electromagnetic, sonic, or other wave energy, preferably in the form of ultrasound, followed by centrifuging the sonicated tissue to form a pellet. In various embodiments, the method is carried out without any enzymatic digestion of the adipose tissue. In other embodiments, the method additionally comprises enzymatically digesting the tissue.

Adipose tissue, like bone marrow, is derived from the embryonic mesenchyme and contains a stroma that is easily isolated. A stem cell population within the adipose stromal compartment can be isolated from adipose tissue such as that derived from liposuction on humans. Like bone marrow derived stem cells, adipose stem cells are capable of differentiating towards the osteogenic, chondrogenic, adipogenic, myogenic, and neurogenic lineages. They are described as multipotent because they are capable of being induced to form a number of cell lineages. Compared with cells harvested from bone marrow, adipose derived stromal cells are easier to obtain and are available in large numbers of stem cells at harvest. In addition, commonly used procedures such as liposuction to remove adipose tissue from patients involve less morbidity or discomfort to the patient than does aspiration of bone marrow.

In various embodiments, the adipose tissue treated by ultrasound or other wave energy is in the form of lipoaspirate that is the product of conventional surgical procedures such as liposuction.

Methods of preparing autologous stem cells from a human or other animal subject are also provided. The methods involve removing adipose tissue from the subject, such as by liposuction or by surgical excision, and exposing the removed tissue to electromagnetic, sonic, or other wave energy such as in the form of ultrasound. After being exposed to ultrasound or other energy, the tissue is centrifuged to form a pellet containing stem cells. The pellet is then implanted into the subject from which the adipose tissue was obtained.

In various embodiments, the method additionally comprises enzymatically digesting the tissue. Digestion may be performed before, during and/or after the subjecting to energy.

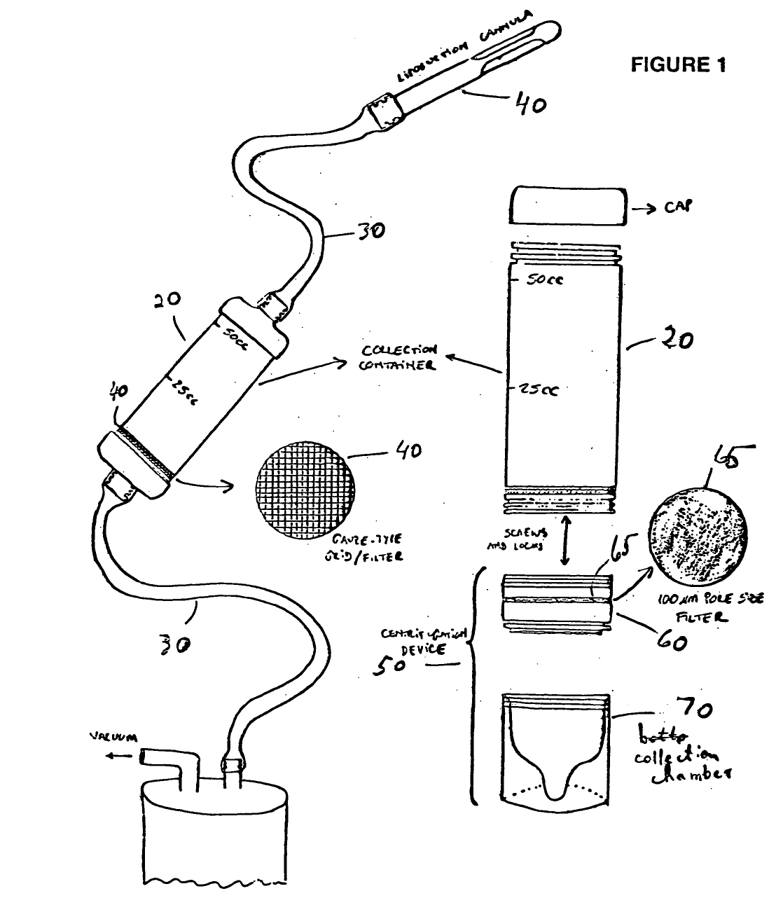

In various embodiments, the tissue is sonicated for less than about 5 minutes and then centrifuged in about 5 minutes. Use of a device such as in FIG. 1 simplifies the steps to be taken, so that cells can be made ready, in various embodiments, for implant in less than about 60 minutes, less than about 30 minutes or less than about 20 minutes. Because of the short time involved, it is feasible to carry out the adipose tissue removal and the pellet composition implanting on the subject in a single intraoperative procedure, saving on medical costs and patient discomfort and inconvenience.

Adult derived adipose stem cells are isolated according to the current teachings from mammals that contain adipose or fat tissue. Fat tissue can be surgically removed from the subcutaneous region of the animal. The current teachings disclose methods of isolating autologous stem cells, meaning cells derived from the same individual that the cells are to be used on as treatment. Autologous cell therapy avoids complications such as tissue availability and problems from immune system rejection and the like.

Human adipose tissue can be obtained from patients undergoing suction-assisted lipectomy (liposuction) or syringe assisted microaspiration procedures according to known techniques. In a typical procedure, carried out under local anesthesia, a hollow, blunt tipped cannula is introduced into the subcutaneous space through small incisions. The cannula is attached to gentle suction and moved through the adipose compartment, mechanically disrupting the fat tissue. A solution of saline and a vasoconstrictor such as epinephrine can be infused into the adipose compartment to minimize blood loss and contamination of the tissue by blood cells. The raw lipoaspirate is collected in a collection chamber such as described further below.

The adipose tissue can be treated with ultrasonic or other wave energy to break down the connective tissue and allow isolation of a fraction containing an increased concentration of stem cells in a subsequent centrifugation step. The wave energy can be applied, for example, in the form of sound waves or as electromagnetic radiation. Electromagnetic radiation such as microwave, infrared, and far infrared can be applied to break up the connective matrix. In various embodiments, electromagnetic radiation is applied to relatively thin sections of adipose tissue to enable the radiation to penetrate throughout the sample being irradiated. Sound waves can be used on larger and thicker samples, as the waves tend to penetrate. In some embodiments, the sound waves contain at least some frequencies at or above about 20,000 Hz. Material exposed to ultrasound or ultrasonic radiation is referred to as being “sonicated”.

In non-limiting embodiments, ultrasonic energy is applied with either a probe sonicator or a bath sonicator. A probe sonicator is inserted into the object being sonicated, while a bath sonicator provides a source of ultrasonic waves that impinges on and travels through the tissue being sonicated. The frequency, power or amplitude, and timing of the application of the ultrasonic energy is selected such that the adipocytes and the connectivity matrix take up the ultrasonic energy and the stem cells in the adipose tissue are not damaged. Conditions of sonication are adjusted until a desirable combination of cell yield, cell viability, and operative time is achieved.

In one embodiment, for example, 50 cc of raw lipoaspirate is sonicated by applying two 30 seconds bursts at 24 kilohertz/60 watts each at room temperature with a 30 second waiting interval between each burst. Immediately after removal from the body, the adipose tissue is at a temperature slightly above normal room temperature. The tissue will cool after removal from the body, and may even be refrigerated or cryopreserved after removal for later use. It is to be understood that sonication conditions can be adjusted, depending on the temperature or state of freezing or thawing of the tissue.

In some embodiments, after sonication, the sonicated tissue is subjected to centrifugation at sufficient speed for a time sufficient to separate and pellet a composition containing the adipose stem cells. In various embodiments, a force of 300 g (i.e. 300 times the force of gravity) for about five minutes is sufficient. In a non-limiting example, the sonicated adipose tissue is centrifuged at about 2000 rpm in a conventional clinical lab centrifuge for about 5 minutes at room temperature.

In some embodiments, following centrifugation, the pellet containing a higher concentration of stem cells is combined with a suitable matrix for further use. In a non-limiting example, the supernatant is decanted and the cellular pellet washed three times with one molar phosphate-buffered saline (PBS). The cellular pellet can then be suspended in a liquid matrix such as saline, PBS, fibrin glue, platelet-rich plasma (PRP), blood, plasma, serum, platelet concentrate, plasma concentrate, or other suitable carrier, including combinations. The suspended pellet can then be implanted directly at sites which need the tissue repair, or alternatively layered onto, infused into, or mixed with a resorbable matrix that can be implanted as needed. Alternatively, the pellet, suspended pellet, or other composition containing the ADAS cells can be chilled or cryopreserved for subsequent use. Further, these ADAS cells can be expanded in number by standard cell culture methods prior to use.

The isolation of a stromal cell fraction containing the stem cells from adipose tissue can be accomplished with any suitable collection and centrifugation devices. A non-limiting example is given in FIG. 1. About 50 cc of adipose tissue is extracted by suction assisted tumescent liposuction inside a specialized collection container 20 attached to suction hoses 30 and to a liposuction cannula 40. The collection container 20 has a gauze-type grid such filter 40 that allows the tumescent fluid to pass through and retains the solid adipose tissue containing the stromal cell population. After collecting the adipose tissue, the collection container 20 is removed from suction hoses 30 and reattached to a centrifugation device 50 containing a filter unit 60 and a rubber receiver 70. The filter unit further contains a 100 micrometer pore size filter 65. Once the collection container 20 containing the adipose tissue is attached to the centrifugation device 50, the tissue is sonicated as described above. After sonication the entire apparatus (i.e. the collection container 20 attached to the centrifugation device 50) is inserted into a centrifuge bucket (not shown) and centrifuged at 300 g for 5 minutes. After centrifugation, the collection container 20 together with the filter unit 60 is detached and can be discarded. The remaining liquid inside the receiver 70 is carefully decanted without disturbing the pellet. The pellet containing adipose stromal cells can then be resuspended in a liquid matrix such as saline, phosphate buffered saline, blood, serum, fibrin glue, platelet-rich plasma, platelet concentrate, plasma concentrate, and the like, as well as combinations.

The methods disclosed here are applicable to any human or other animal species. In various embodiments, the methods comprise the derivation of human adipose stem cells. In other embodiments, the methods comprise the derivation of non-human adipose stem cells.

In various embodiments, intraoperative methods for treating a patient or subject with chronic or acute soft tissue injury are provided. The methods include removing fat tissue from the subject as described above, and exposing the fat tissue to ultrasonic energy. Optionally, the tissue may be subjected to enzymatic digestion. The sonicated tissue is then centrifuged to form a pellet containing multipotent cells. The pellet is suspended in a liquid matrix and the suspended pellet is applied to the site of the injury. In various embodiments, the suspended pellet containing the stem cells is implanted directly at sites in need of tissue repair or layered onto a resorbable matrix that can be implanted as needed. The liquid matrix into which the pellet is suspended can contain conventional biological fluids such as saline, phosphate-buffered saline, blood platelets, fibrin glue, plasma or serum, blood, platelet concentrate, plasma concentrate, and the like.

Accordingly, in various embodiments, the present invention provides compositions for tissue construction in a human or other animal subject, comprising:
(a) adipose derived stem cells; and
(b) a biocompatible carrier;
   wherein said adipose derived stem cells are derived by application of electromagnetic, sonic, or other wave energy to adipose tissue. In various embodiments, the stem cells are further derived by enzymatic digestion. As referred to herein, a “biocompatible carrier” is a material that contains or supports stem cells, preferably enabling their growth at the site of implantation. The nature of the carrier will depend upon the specific site of implantation of the stem cells. In one embodiment, the stem cells are mixed with the carrier prior to implantation. In other embodiments, the scaffold material is implanted before and/or after the stem cells are implanted. Suitable carrier materials include porous or semi-porous, natural, synthetic or semi-synthetic materials.

In various embodiments for application to bony tissue, the carrier is an osteoconductive material. Scaffold materials include those selected from the group consisting of bone (including cortical and cancellous bone), demineralized bone, ceramics, polymers, metals, and combinations thereof. Ceramics include any of a variety of ceramic materials known in the art for use for implanting in bone, including calcium phosphate (including tricalcium phosphate, tetracalcium phosphate, hydroxyapatite, and mixtures thereof. Polymers include collagen, gelatin, polyglycolic acid, polylactic acid, polypropylenefumarate, and copolymers or combinations thereof. A preferred ceramic is commercially available as ProOsteon™ from Interpore Cross International, Inc. (Irvine, Calif., U.S.A.).

The present disclosure also provides methods for tissue construction in human or non-human animals comprising the use of adipose stem cells derived by applying electromagnetic, sonic, or other wave energy to adipose tissue. In various embodiments, the stem cells are further derived by enzymatic digestion. Methods of tissue construction include cosmetic and therapeutic procedures. Therapeutic procedures include those for the repair of chronic or acute hard or soft tissue injuries that can be treated by the method, such as surgical incisions, diabetic ulcers, bed sores, and chronic venous insufficiency wounds.

Advantageously, the steps of removing adipose tissue from the patient to suspending the recovered pellet in a liquid matrix and applying the suspended pellet to a wound can be accomplished in a relatively short period of time. This allows for the removal of the fat tissue and applying the suspended pellet including stem cells to the site of injury to be accomplished in a single operative procedure.

### EXAMPLE 1

### Comparative

A stromal vascular fraction is isolated from 50 cc of raw human lipoaspirate according to established methodology. The lipoaspirate is washed extensively with equal volumes of phosphate-buffered saline (PBS), and the extracellular matrix is digested at 37° C. for 30 minutes with 0.075 percent collagenase. After digestion, enzyme activity is neutralized with Dulbecco's modified Eagle's medium (DMEM) containing 10 percent FBS (fetal bovine serum) and centrifuged at 1200 g for 10 minutes to obtain a high-density pellet. The pellet is resuspended in 160 mM NH₄Cl and incubated at room temperature for 10 minutes to lyse contaminating red blood cells. The stromal vascular fraction is collected by centrifugation at 1200 g, filtered through a 100 micrometer nylon mesh to remove cellular debris and incubated overnight at 37° C. in an atmosphere of 5 percent CO₂ and a control medium (DMEM, 10 percent FBS, 1 percent antibiotic/antimycotic solution). The procedure is described in Zuk et al., Tissue Engineering, Vol. 7, pg. 211-228.

### EXAMPLE 2

### Isolation of Stromal Cells from Adipose Tissue Using a Two Step Sonication/Centrifugation Method

50 cc of raw lipoaspirate, extracted by suction assisted liposuction or syringe assisted microaspiration, is loaded into a conical tube. Using either a probe sonicator or bath sonicator, the adipose tissue is liquefied by applying two 30 seconds bursts at 24 kilohertz/60 watts each at room temperature with 30 second waiting intervals between each burst. The conical tube is then capped and the sonicated adipose tissue is centrifuged at 2000 rpm for 5 minutes at room temperature in a clinical centrifuge. Following centrifugation, the supernatant is decanted and the cellular pellet is washed 3 times with 50 milliliters of 1 molar phosphate-buffered saline (PBS). The cells contained in this pellet when cultured in a culture medium such as DMEM or Ham's F12 supplemented with fetal calf serum develop a fibroblast-like or stellate morphology typical of mesenchymal stem cells.

What is claimed is:

1. A method for isolating or recovering adult stem cells from adipose tissue composing:
subjecting adipose tissue to an electromagnetic or sonic energy source; and
centrifuging the tissue to form a pellet comprising stem cells.

2. A method according to claim 1, wherein the method does not comprise an enzymatic digestion step.

3. A method according to claim 1, additionally comprising enzymatic digestion of the tissue.

4. A method according to claim 1, wherein the energy source is electromagnetic.

5. A method according to claim 1, wherein the energy source is sonic.

6. A method for preparing autologous stem cells from a human patient comprising:
removing adipose tissue by liposuction;
exposing the removed tissue to ultrasound; and
centrifuging the sonicated tissue to form a pellet comprising the stem cells.

7. A method according to claim 6, wherein the method does not comprise an enzymatic digestion step.

8. A method according to claim 6, additionally comprising enzymatic digestion of the tissue.

9. An intraoperative method for treating a human or other animal subject having a chronic or acute soft tissue injury comprising:
removing fat tissue from the subject;
exposing the fat tissue to ultrasonic energy;
centrifuging the sonicated tissue to form a pellet comprising multipotent cells;
suspending the pellet in a liquid matrix; and
applying the suspended pellet to the site of the injury.

10. A method according to claim 9, wherein the liquid matrix comprises saline.

11. A method according to claim 10, wherein the liquid matrix comprises phosphate-buffered saline.

12. A method according to claim 9, wherein the liquid matrix comprises concentrated blood platelets.

13. A method according to claim 9, wherein the liquid matrix comprises blood.

14. A method according to claim 9, wherein the liquid matrix comprises serum or plasma.

15. A method according to claim 9, wherein the injury is an acute surgical wound.

16. A method according to claim 9, wherein removing the fat tissue comprises liposuction.

17. A method according to claim 9, wherein all of the steps are carried out on the subject during a single operation.

18. A method according to claim 9, wherein the subject is human.

19. A composition for tissue construction in a human or other animal subject, comprising:
(a) adipose derived stem cells; and
(b) a biocompatible carrier;
wherein said adipose derived stem cells are derived by application of electromagnetic or sonic energy to adipose tissue.

20. A method according to claim 19, wherein the energy source is ultrasonic.
